(19) United States
(12) Patent Application Publication
(10) Pub. No.: US 2024/0164262 A1
(43) Pub. Date: May 23, 2024

(54) METHOD AND APPARATUS FOR CONTROLLING AERIAL MYCELIUM GROWTH BY ELECTRONIC MIST DETECTION

(71) Applicant: Ecovative Design LLC, Green Island, NY (US)

(72) Inventors: Peter James Mueller, Poestenkill, NY (US); Asa Trench Snyder, Petersburg, NY (US); Ian Thomas Bonesteel, Wynantskill, NY (US); Chirayu Jain, Troy, NY (US); Eben D. Bayer, Troy, NY (US); Alex Stephen Friedman, Troy, NY (US)

(21) Appl. No.: 18/514,792

(22) Filed: Nov. 20, 2023

Related U.S. Application Data
(60) Provisional application No. 63/427,074, filed on Nov. 21, 2022.

(51) Int. Cl. A01G 18/69; A01G 18/20; A01G 18/62

(52) U.S. Cl. CPC A01G 18/69; A01G 18/20; A01G 18/62

(57) ABSTRACT

Apparatus and processes to grow a biomaterial made of aerial mycelium by sensing and controlling airborne mist concentration, regardless of relative humidity. A growth matrix comprising a growth medium and a fungus, is grown under controlled environmental conditions to produce a mycelium product. To control growth conditions precisely and efficiently, airborne mist is electronically detected using one or more sensors, configured to measure airborne mist concentration visually, optically, chemically, electromagnetically, or by laser, ultrasonically, with radar, or other means. Electronic detection of airborne mist using one or more sensors generates a signal that is transmitted to a processor that can either maintain, increase, or decrease airborne mist concentration in a growth environment. The present invention provides processes of growing mycelium that are repeatable and resource efficient, while providing high quality and quantity mycelium-based products.

### CROSS-REFERENCE TO RELATED APPLICATIONS

This application is related to U.S. Provisional Patent Application No. 63/427,074, filed Nov. 21, 2022, entitled “METHOD AND APPARATUS FOR CONTROLLING AERIAL MYCELIUM GROWTH BY ELECTRONIC MIST DETECTION,” the disclosure of which is incorporated herein by reference in its entirety.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 C.F.R. § 1.57.

### BACKGROUND

### Field

This application relates generally to improved apparatus and processes of growing aerial mycelium products, and in particular, for sensing moisture concentrations to control conditions within a mycelial growth environment more precisely and efficiently.

### Description

Environmentally friendly alternatives to traditional material production are in high demand, either in the food industry (e.g., meat substitutes) or in non-food-related industries, such as textiles, packaging, construction, and other industries. Products made from fungal mycelia fill such demand. As a result, fungal mycelia are increasingly used as an efficient and biodegradable material across several industrial applications.

The growing demand for and relative novelty of mycelium-based products has resulted in a parallel need for fungal tissue growing methods that are reproducible and energy efficient. In general, there is a growing need for large-scale methods for growing mycelium-based products that yield quality tissue for cost-effective industrial application. One such means of achieving cost-effective and quality yield is by precisely controlling inputs to the mycelium growing process.

### SUMMARY

For purposes of summarizing the invention and the advantages achieved over the prior art, certain objects and advantages of the invention have been described herein. Of course, it is to be understood that not necessarily all such objects or advantages may be achieved in accordance with any particular embodiment of the invention. Thus, for example, those skilled in the art will recognize that the invention may be embodied or carried out in a manner that achieves or optimizes one advantage or group of advantages as taught herein without necessarily achieving other objects or advantages as may be taught or suggested herein.

In a first aspect, a method for growing mycelium comprises the steps of providing a growth matrix within a growth environment, and electronically detecting the concentration of airborne mist within the portion of the growth environment. In various aspects, the process can include a growth matrix comprising a growth medium and a fungus. The method can be implemented to form an aerial mycelium biomaterial, wherein the aerial mycelium biomaterial can comprise, consist essentially of, or consist of fungal mycelium. In various aspects, the portion of the growth environment can comprise between 0.01% and 100% of the growth environment total volume.

In various aspects, the electronic detection of airborne mist concentrations can be configured to determine the absolute or relative amount of airborne liquid (e.g., droplets). The step of electronically detecting airborne mist can be made via a sensor, such as a visual, optical, laser, chemical, ultrasonic, radar, camera, particle flow imaging, photodetector, conductivity sensor, particle sizer, microwave, radio frequency (RF) sensor, near-infrared or infrared sensor (or any type of sensor on the electromagnetic spectrum, e.g., ultraviolet to RF), LED, fog, electromagnetic, or other type of sensor. For example, the concentration or density of airborne droplets can be determined based on the diameter of a liquid droplet or on the chemical signature of airborne liquids. Droplets can be further measured by their size, size distribution, droplet number per given area, deposition rate and/or velocity in any direction. As a further example, a sensor may determine the chemical signature of the liquid (e.g., water), or an element contained in the liquid droplets. In various further aspects, a dye could be placed within the liquid that forms the liquid droplets for spectral detection by any of the above exemplified means. Alternatively, nutrients may be placed within the liquid that forms the liquid droplets for spectral detection. Still in a further alternative embodiment, substances may be placed within the liquid droplets that discourage growth of non-desired organisms within aerial mycelium, and which also demonstrate unique and detectable spectral signatures. Airborne liquids (or dyes contained therein, as an example) should be inert such that they do not have a negative impact on mycelium growth or development, and do not pose any negative impact for human consumption, epidermal contact, or for use in food applications and textile applications.

In various aspects, the sensor can be placed in a central position within the growth environment. However, the sensor can be placed in a plurality of positions, either within or outside the growth environment (but in close association with the growth environment to allow for continuous sensing of airborne liquid droplets). The sensor can be placed in the center of the growth environment or at any position within the growth environment, depending on, for example, the desired specificity of airborne mist concentration measurements of particular locations within a growth environment. Additionally, more than one sensor can be used within or outside a growth environment. For example, a growth environment in which there is a plurality of shelves that vertically house inoculated substrate can have one or more sensors located along a central aisle between rows of vertically stacked shelving, and/or along an outer edge of the outermost vertically stacked shelve(s). Alternatively, one or more sensors can be placed on or near each shelf or rack for more localized monitoring of airborne mist concentrations. In some embodiments, one or more sensors can be placed on a low, e.g., lowest, position within the growth environment. Further examples of sensor locations inside the growth environment include: the floor, suspended from a wall or ceiling, or suspended from or positioned on one or more shelves or growth areas within the growth environment. Further examples of sensor locations outside of the growth environment include: outside of a viewing window, or within an air handler for the growth environment, and/or in any other locations. The sensor(s) can, in one embodiment, be capable of detecting a concentration value of airborne mist within the growth environment within a distance (e.g., radius) from the sensor of between about 0.01 in to about 500 ft, and in some embodiments, between about 1.0 in to about 250 ft.

In another aspect, a sensor is in communication with a processor. The sensor is in communication with the processor to transmit information related to airborne mist concentration, for example, that there is too much mist in the growth environment or portions thereof. In response, the processor can be configured to either reduce mist input into the growth environment or to turn off the mist apparatus altogether. The same is true for examples involving too little mist in a growth environment; the processor can be configured to either increase mist input or to turn on the mist apparatus. Where the sensor communicates that airborne mist concentration is already at the desired level, the processor may transmit a signal, for example, that tells the mist apparatus to make no changes to mist input. In some further aspects, a sensor and processor can communicate directly to a misting apparatus, or through a central processing unit. For example, a central processing unit may be used to modulate or pulse airborne mist concentration such that misting occurs at select intervals during the growth cycle of an inoculated substrate.

In some aspects, electronic detection of airborne mist concentration is envisioned as a method to more efficiently allocate resources, thereby saving on, for example, water and/or other liquid usage in larger commercial contexts. Electronic detection of airborne mist concentration may entail direct control of the misting apparatus by the sensor, or indirect control through a central processing system. In some further aspects, multiple types of sensors may be employed to determine the concentration of airborne liquid of more than one type of liquid, and/or to determine the level of airborne liquid droplets that have not yet evaporated in the overall growth environment.

In another aspect, an apparatus for growing mycelium is described. For example, such apparatus may include one or more airborne liquid droplet detection devices designed to detect airborne liquid through visual inspection (along the human visual spectrum). In a further embodiment, such apparatus may include an airborne mist detection device designed to detect airborne liquid through a means other than through use of measurement along the human visual spectrum. In a further embodiment, such apparatus may include one or more cameras in conjunction with a processing unit.

In various aspects, the apparatus for growing mycelium is used to grow mycelium from a growth matrix comprising a growth medium and a fungus. In some further aspects, the apparatus may include a growth environment in which the mycelium is grown. The growth environment is comprised of a total volume that can be divided into portions. In some embodiments, a portion of the growth environment can have a volume that is between 0.01% to 100% of the growth environment total volume.

In various aspects, the apparatus for growing mycelium comprises one or more sensors that may be configured to generate a first signal that is indicative of the airborne mist concentration within a growth environment. The first signal is then electronically communicated to a processor. A processor can receive the first signal to control airborne mist concentration in the growth environment in response to the first signal. The processor is configured to increase or decrease mist concentration in the growth environment if the first signal indicates a mist concentration in the growth environment that is below or above, respectively, a predetermined set point. The processor is further configured to do nothing if the current mist concentration in the growth environment is at the desired level

In various aspects, the first signal that is indicative of the airborne mist concentration within a growth environment is a measure of the interaction between light and an airborne mist droplet. The first signal can be the product of a variety of electromagnetic-based methods for mist detection which rely on the principle that a liquid particle will interact with, and diffuse or reflect incoming electromagnetic radiation.

In various aspects, mist is introduced into the growth environment through at least one inlet. In some embodiments, at least one inlet may be configured to introduce aqueous mist into the portion of the growth environment.

In various aspects, the mist sensor can be a near-infrared or infrared sensor, a fog sensor, a camera and/or an altered camera. Airborne mist concentration or density can be monitored visually, optically, by near-infrared or infrared, by laser, chemically, ultrasonically, with radar, LED, or by others means. The sensor can be programmed to be a source of, respond to, and monitor electromagnetic radiation. The sensor may further monitor airborne mist concentration and/or density either continuously or at scheduled intervals of any length of time. Scheduled intervals can be adjusted if the first signal is erroneous, inaccurate, corrupted, or otherwise incomplete either manually or automatically. In an alternative embodiment, various, similar or alternative mist sensors may be employed within a growth environment, and arranged in a grid-like configuration in order to encompass a pre-determined amount of the growth environment space, or in a configuration that provides for cross-checking airborne mist concentration levels in particular regions of a growth environment space.

All of these embodiments are intended to be within the scope of the invention herein disclosed. These and other embodiments of the present invention will become readily apparent to those skilled in the art from the following detailed description of the preferred embodiments having reference to the attached figures, the invention not being limited to any particular preferred embodiment(s) disclosed.

### BRIEF DESCRIPTION OF THE DRAWINGS

The features and advantages of the methods and compositions described herein will become more fully apparent from the following description and appended claims, taken in conjunction with the accompanying drawings. These drawings depict only several embodiments in accordance with the disclosure and are not to be considered limiting of their scope. In the drawings, similar reference numbers or symbols typically identify similar components, unless context dictates otherwise. In some instances, the drawings may not be drawn to scale.

FIG. 1A illustrates an embodiment of a growth matrix suitable to support extra-particle aerial mycelial growth.

FIG. 1B illustrates an embodiment of extra-particle aerial mycelial growth extending from the growth matrix of FIG. 1A.

FIG. 2 illustrates an isometric view of a system for cultivating aerial mycelia.

FIGS. 3A and 3B illustrate a top and front view, respectively, of an embodiment of a system configured to electronically detect an airborne mist concentration value within a growth environment.

FIG. 4 illustrates a flow diagram of an embodiment of a method for growing mycelium.

FIG. 5 illustrates a lateral view of a mounted sensor configured to generate signals indicative of airborne mist concentration in a growth environment.

FIG. 6 illustrates a flow diagram of an embodiment of control logic for detecting and controlling airborne mist.

FIGS. 7A and 7B illustrate a top and front view, respectively, of an embodiment of a system configured to electronically detect an airborne mist concentration value in a plurality of portions of the growth environment using multiple sensors.

### DETAILED DESCRIPTION

U.S. Patent Application Publication No. 2015/0033620, International PCT Patent Application No. WO2019/099474A1, the entirety of which is incorporated herein by reference thereto, except where inconsistent with the disclosure herein, describes systems and methods of growing and/or harvesting a mycological biopolymer material and products resulting therefrom.

Deposition of a controlled amount of mist within a growth environment can be important for reliable and tunable production of aerial mycelium. The mist deposition rate has typically been directly measured using physical collection and weighing. However, this process takes significant time (e.g., 24 hours or more) to generate a detectable signal. As a result, weighing deposition falls short of providing real-time feedback for system controls, e.g., to increase or decrease misting rates.

Conventional mycelium growing methods typically use a fixed mister rate, which is calibrated and pre-determined prior to operation, or use a highly sensitive relative humidity probe, which gives a live reading that is imprecisely correlated with actual deposition. Each of these methods have significant limitations.

The first method, fixed mister rates, has two key limitations. First, it is calibrated before the growth environment is loaded with mycelium growing material. Consequently, if there is any interaction between the biomass and the resulting deposition rate, the calibration will be inaccurate. As the amount of biomass placed in the growth environment increases, the less accurate growth environment calibration will be when it takes place prior to the placement of biomass in the empty growth environment. Miscalibration results in a mismatch between the calibrated mist deposition rate and the amount of mist actually deposited. Second, a fixed deposition rate, by definition, does not allow for adjustments over the course of the mycelium growth and the growth environment operation. Metabolic rates and other thermodynamic factors that affect the amount of mist desired may not be stable, and a fixed mist deposition rate could not adapt to these changes.

The second method, relative humidity control, is unreliable in monitoring consistent performance of deposition rates. Relative humidity is a measure of water vapor (i.e., a gaseous form of water); in typical operating conditions, the growth environment is fully saturated with water vapor. Airborne water droplets (i.e., water droplets in liquid form, distinguished from water in vapor form) exist in suspension and increase the total water capacity of the air volume beyond the water that exists in fully saturated vapor. Relative humidity probes are not designed to measure this phenomenon. Although relative humidity probes can be operated above their calibration range (e.g., above 99% relative humidity) and used to control misting, any resulting relative humidity measurements do not represent true relative humidity levels. Thus, these measurements are not particularly accurate to use as reliable control signals. Thus, this method has been shown to be particularly susceptible to error based on sensor choice, making it unreliable for commercial production.

The present application describes a process and apparatus that use detection (e.g., visibility) within the growth environment of airborne mist (e.g., airborne liquid water) content within the air, and controls misting and mist concentration levels within the growth environment based on real-time, live measurements of airborne droplet deposition rates relative to accurate and reliable pre-calibration. This can allow the growth environment to be maintained within a stable, steady state desired mist concentration level, even when, for example, the growth environment is in a nearly saturated, or fully saturated state (e.g., 99% or greater, or even 100% humidity), with additional airborne mist (e.g., suspended liquid water) content. The process and apparatus herein can be implemented to accurately detect the presence of airborne mist and/or airborne mist concentration levels in a growth environment, for example, with a sensor, regardless of the presence of vapor (e.g., water vapor) and/or regardless of the level of relative humidity. In some embodiments, the process and apparatus can detect the presence of airborne mist and/or airborne mist concentration levels in a separate step from detecting, or without detecting, the presence or amount of vapor and/or relative humidity levels. In some embodiments, a sensor that is not a relative humidity sensor is implemented. In some embodiments, a sensor that configured only to sense the presence of airborne mist and/or the airborne mist concentration level is implemented

Visibility can be measured using a wide range of techniques, including with the human eye and charts at a fixed distance. In a more reliable embodiment, a “fog sensor,” such as one comprising a near-infrared or infrared LED coupled with a phototransistor tuned to respond to near-infrared or infrared wavelengths, is used to give a novel and unexpectedly reliable and consistent real time measurement of the optical density of the fog within a growth environment. In another embodiment, a “fog sensor,” such as is used in meteorological, air traffic, or maritime contexts, is used to give a novel and unexpectedly reliable and consistent real time measurement of the optical density of the fog within a growth environment. Some nonlimiting examples of fog sensors include the FD410-MINI, MinPWS, MiniOFS, and PWD920A2, available from companies and retailers such as Optical Sensors Sweden AB, Alibaba, Pharos Marine Automatic Power, Inc., or other companies. Once optical density has been calibrated against liquid (e.g., water) deposition, it can be used to live-adjust misting rates to maintain an ideal deposition rate. Optical density is highly sensitive to changes, allowing very precise control of deposition ranges. It is also adaptable to changes in the thermodynamics or organism metabolism during growth environment operation. Additionally, it can allow the growth environment to operate under a single control paradigm, regardless of the amount of material loaded into the growth environment.

The following discussion presents detailed descriptions of the several embodiments of apparatus or processes for growing aerial mycelium shown in the figures. These embodiments are not intended to be limiting, and modifications, variations, combinations, etc., are possible and within the scope of this disclosure.

The present disclosure provides for processes for growing aerial mycelium and apparatus for enabling processes for growing aerial mycelium.

It is an object of the invention to provide an improved process and apparatus that involves electronic detection of airborne mist concentration within a growth environment.

It is yet another object of the invention to provide a process and apparatus for growing aerial mycelium that includes electronically detecting airborne mist concentration within a growth environment.

It is yet another object of the invention to provide a process further comprising controlling the airborne mist concentration within a growth environment in response to an airborne mist concentration value.

It is yet another object of the present invention to provide a process of controlling the airborne mist concentration within a growth environment by either increasing, decreasing or maintaining the mist concentration in a growth environment.

It is yet another object of the present invention to provide a process of detecting airborne mist concentration, comprising detection by one or more of visual, optical, near-infrared or infrared, laser, chemical, ultrasonic, radar, LED, or other means. Detection can be implemented using a camera (e.g., infrared camera). Detection can be accomplished without detecting relative humidity.

It is yet another object of the present invention to provide a process of measuring airborne mist droplets by determining their size, diameter, size distribution, number per unit area, velocity and/or deposition rate. For example, alternative variations of the sensing apparatus might include particle size measurement capability, average diameter measurements, particle size distribution measurements, which could then be used to direct particular liquid (e.g., water) particle sizes desired, or size distributions desired. Such variations in sizes or distributions could then be directed to particular regions (e.g., shelves and/or racks, or portions thereof) in a growth chamber.

It is yet another object of the present inventor to provide a process of detecting and controlling airborne mist concentration either continuously or during scheduled time intervals during the growth cycle (e.g., pulsed or modulated). For example, misting in select intervals may be more suitable to particular organism growth, or more efficient from a resource perspective.

It is another object of the invention to provide an apparatus for growing mycelium, an aerial mycelium product, and/or processes of making mycelium-based products comprised wholly or in part of fungal hyphae.

It is yet another object of the invention to provide an apparatus for growing mycelium that can be placed inside or outside of a growth environment configured to grow mycelium from a growth matrix.

It is yet another object of the invention to provide an apparatus for growing mycelium that comprises a sensor. The sensor is configured to generate a first signal indicative of an airborne mist concentration value within a growth environment, which is then communicated to a processor configured to either increase, decrease, or maintain airborne mist concentration in a growth environment. If the first signal is somehow incomprehensible by the processor, time intervals during which a signal is generated by the sensor can be adjusted manually or automatically.

It is yet another object of the invention to provide an apparatus for growing mycelium that comprises a sensor for detecting airborne mist concentration using visual, optical, near-infrared or infrared, laser, chemical, ultrasonic, radar, or other means.

### Definitions

The aerial mycelia of the present disclosure are growth products obtained from a growth matrix incubated for a period of time (i.e., an incubation time period) in a growth environment, as disclosed herein.

“Mycelium” as used herein refers to a connective network of fungal hyphae, with mycelia being the plural form of mycelium.

“Hyphae” as used herein refers to branched filament vegetative cellular structures that are interwoven to form mycelium.

“Fruiting body” as used herein refers to a fungal stipe, pileus, gill, pore structure, or a combination thereof, and may be referred to herein as “mushroom.”

“Substrate” as used herein refers to a material or surface thereof, from or on which an organism lives, grows, and/or obtains its nourishment. In some embodiments, a substrate provides sufficient nutrition to the organism under target growth conditions such that the organism can live and grow without providing the organism a further source of nutrients. A variety of substrates are suitable to support the growth of an aerial mycelium of the present disclosure. Suitable substrates are disclosed, for example, in US20200239830A1, the entire contents of which are hereby incorporated by reference in their entirety to the extent not inconsistent with the content of this disclosure. In some embodiments, the substrate is a natural substrate. Non-limiting examples of a natural substrate include a lignocellulosic substrate, a cellulosic substrate, or a lignin-free substrate. A natural substrate can be an agricultural waste product or one that is purposefully harvested for the intended purpose of food production, including mycelial-based food production. Further non-limiting examples of substrates suitable for supporting the growth of mycelia of the present disclosure include soy-based materials, oak-based materials, maple-based materials, corn-based materials, seed-based materials and the like, or combinations thereof. The materials can have a variety of particle sizes, as disclosed in US20200239830A1, and occur in a variety of forms, including shavings, pellets, chips, flakes, or flour, or can be in monolithic form. Non-limiting examples of suitable substrates for the production of mycelia of the present disclosure include corn stover, maple flour, maple flake, maple chips, soy flour, chickpea flour, millet seed flour, oak pellets, soybean hull pellets and combinations thereof. Additional useful substrates for the growth of mycelia are disclosed herein.

“Growth media” or “growth medium” as used herein refers to a matrix containing a substrate and an optional further source of nutrition that is the same or different than the substrate, wherein the substrate, the nutrition source, or both are intended for fungal consumption to support mycelial growth.

“Growth matrix” as used herein refers to a matrix containing a growth medium and a fungus. In some embodiments, the fungus is provided as a fungal inoculum; thus, in such embodiments, the growth matrix comprises a fungal-inoculated growth medium. In other embodiments, the growth matrix comprises a colonized substrate.

“Inoculated substrate” as used herein refers to a substrate that has been inoculated with fungal inoculum. For example, an inoculated substrate can be formed by combining an uninoculated substrate with a fungal inoculum. An inoculated substrate can be formed by combining an uninoculated substrate with a previously inoculated substrate. An inoculated substrate can be formed by combining an inoculated substrate with a colonized substrate.

“Colonized substrate” as used herein refers to an inoculated substrate that has been incubated for sufficient time to allow for fungal colonization. A colonized substrate of the present disclosure can be characterized as a contiguous hyphal mass grown throughout the entirety of the volume of the growth media substrate. The colonized substrate may further contain residual nutrition that has not been consumed by the colonizing fungus. As is understood by persons of ordinary skill in the art, a colonized substrate has undergone primary myceliation, sometimes referred to by skilled artisans as having undergone a “mycelium run.” Thus, in some particular aspects, a colonized substrate consists essentially of a substrate and a colonizing fungus in a primary myceliation phase. For many fungal species, asexual sporulation occurs as part of normal vegetative growth, and as such could occur during the colonization process. Accordingly, in some embodiments, a colonized substrate of the present disclosure may also contain asexual spores (conidia). In some aspects, a colonized substrate of the present disclosure can exclude growth progression into sexual reproduction and/or vegetative foraging. Sexual reproduction includes fruiting body formation (e.g., primordiation and differentiation) and sexual sporulation (meiotic sporulation). Vegetative foraging includes any mycelial growth away from the colonizing substrate (such as aerial growth). Thus, in some further aspects, a colonized substrate can exclude mycelium that is in a vertical expansion phase of growth. A colonized substrate can enter a mycelial vertical expansion phase during incubation in a growth environment of the present disclosure. For example, a colonized substrate can enter a mycelial vertical expansion phase upon introducing aqueous mist into the growth environment and/or depositing aqueous mist onto colonized substrate and/or any ensuing extra-particle growth. In some embodiments, the use of aqueous mist can be adjusted, for example, to desired levels and timing, to affect the topology, morphology, density, and/or volume of the growth.

Any suitable substrate can be used alone, or optionally combined with a nutrient source, as media to support mycelial growth. The growth media can be hydrated to a final target moisture content prior to inoculation with a fungal inoculum. In a non-limiting example, the substrate or growth media can be hydrated to a final moisture content of at least about 50% (w/w), at most about 95% w/w, within a range of about 50% to about 95%, about 50% to about 90%, about 50% to 85%, about 50% (w/w) to about 80% (w/w), about 50% (w/w) to about 75% (w/w), within a range of about 50% (w/w) to about 65% (w/w), within a range of about 50% (w/w) to about 60% (w/w), or within a range of about 60% (w/w) to about 70% (w/w). Growth media hydration can be achieved via the addition of any suitable source of moisture. In a non-limiting example, the moisture source can be airborne or non-airborne liquid phase water (or other liquids), an aqueous solution containing one or more additives (including but not limited to a nutrient source), and/or gas phase water (or other compound). In some embodiments, at least a portion of the moisture is derived from steam utilized during bioburden reduction of the growth media. In some embodiments, inoculation of the growth media with the fungal inoculum can include a further hydration step to achieve a target moisture content, which can be the same or different than the moisture content of the growth media. For example, if growth media loses moisture during fungal inoculation, the fungal inoculated growth media can be hydrated to compensate for the lost moisture.

Methods for the production of aerial mycelium disclosed herein can include an inoculation stage, wherein an inoculum is used to transport an organism into a substrate. The inoculum, which carries a desired fungal strain, is produced in sufficient quantities to inoculate a target quantity of substrate. The inoculation can provide a plurality of myceliation sites (nucleation points) distributed throughout the substrate. Inoculum can take the form of a liquid, a slurry, or a solid, or any other known vehicle for transporting an organism from one growth-supporting environment to another. Generally, the inoculum comprises water, carbohydrates, sugars, vitamins, other nutrients, and fungi. The inoculum may contain enzymatically available carbon and nitrogen sources (e.g., lignocellulosic biomass, chitinous biomass, carbohydrates) augmented with additional micronutrients (e.g., vitamins, minerals). The inoculum can contain inert materials (e.g., perlite). In a non-limiting example, the fungal inoculum can be a seed-supported fungal inoculum, a feed-grain-supported fungal inoculum, a seed-sawdust mixture fungal inoculum, or another commercially available fungal inoculum, including specialty proprietary spawn types provided by inoculum retailers. In some aspects, a fungal inoculum can be characterized by its density. In some embodiments, a fungal inoculum has a density of about 0.1 gram per cubic inch to about 10 grams per cubic inch, or from about 1 gram per cubic inch to about 7 grams per cubic inch. A skilled person can modify variables including the substrate or growth media component identities, substrate or growth media nutrition profile, substrate or growth media moisture content, substrate or growth media bioburden, inoculation rate, and inoculum constituent concentrations to arrive at a suitable medium to support aerial mycelial growth. In some embodiments, the inoculation rate can be expressed as a percentage of the target volume of the substrate or growth media (% (v/v)). In some embodiments, the inoculation rate can range from about 0.1% (v/v) to about 80% (v/v). In some embodiments, the inoculation rate is at most about 50% (v/v), at most about 45% (v/v), at most about 40% (v/v), at most about 30% (v/v), at most about 25% (v/v), at most about 20% (v/v), at most about 15% (v/v), at most about 10% (v/v) or at most about 5% (v/v). In some embodiments, the inoculation rate is about 1% (v/v), about 2% (v/v), about 3% (v/v), about 4% (v/v), about 5% (v/v), about 6% (v/v), about 7% (v/v), about 8% (v/v), about 9% (v/v), about 10% (v/v), about 11% (v/v), about 12% (v/v), about 13% (v/v), about 14% (v/v), about 15% (v/v), about 16% (v/v), about 17% (v/v), about 18% (v/v), about 19% (v/v), about 20% (v/v), about 21% (v/v), about 22% (v/v), about 23% (v/v), about 24% (v/v), about 25% (v/v), about 26% (v/v), about 27% (v/v), about 28% (v/v), about 29% (v/v) or about 30% (v/v); or any range therebetween. In some embodiments, the inoculation rate can be expressed as a percentage of the target dry mass of the substrate or growth media (% (w/w)). In some embodiments, the inoculation rate can range from about 0.1% (w/w) to about 80% (w/w). In some embodiments, the inoculation rate is at most about 50% (w/w), at most about 45% (w/w), at most about 40% (w/w), at most about 30% (w/w), at most about 25% (w/w), at most about 20% (w/w), at most about 15% (w/w), at most about 10% (w/w) or at most about 5% (w/w). In some embodiments, the inoculation rate is about 1% (w/w), about 2% (w/w), about 3% (w/), about 4% (w/w), about 5% (w/w), about 6% (w/w), about 7% (w/w), about 8% (w/w), about 9% (w/w), about 10% (w/w), about 11% (w/w), about 12% (w/w), about 13% (w/w), about 14% (w/w), about 15% (w/w), about 16% (w/w), about 17% (w/w), about 18% (w/w), about 19% (w/w), about 20% (w/w), about 21% (w/w), about 22% (w/w), about 23% (w/w), about 24% (w/w), about 25% (w/w), about 26% (w/w), about 27% (w/w), about 28% (w/w), about 29% (w/w) or about 30% (w/w); or any range therebetween.

“Growth environment” as used herein refers to an environment that supports the growth of mycelia, as would be readily understood by a person of ordinary skill in the art in the mycelial cultivation industry, which contains a growth atmosphere having a gaseous environment of carbon dioxide (CO₂), oxygen (O₂), and a balance of other atmospheric gases including nitrogen (N₂), and which is further characterized as having a relative humidity. In some aspects of the present disclosure, the growth atmosphere can have a CO₂ content of at least about 0.02% (v/v), at least about 0.6%, at least about 5% (v/v), less than about 10% (v/v), less than about 8% (v/v), less than about 7%, between about 0.02% and 10%, between about 0.02% and 8%, between about 0.6% and about 7%, between about 5% and about 10%, or between about 5% and about 8%. In some other aspects, the growth atmosphere can have an O₂ content of at least about 12% (v/v), or at least about 14% (v/v), and at most about 21% (v/v). In yet other aspects, the growth atmosphere can have an N₂ content of at most about 79% (v/v). Each foregoing CO₂, O₂ or N₂ content is based on a dry gaseous environment, notwithstanding the growth environment atmosphere relative humidity. “A portion of the growth environment” as used herein refers to a percentage of the total volume of the growth environment. For example, a portion of the growth environment can encompass between 0.01% to 100% of the total volume of the growth environment. A portion of the growth environment can refer to any fraction of the one-dimensional, two-dimensional or three-dimensional geometry comprising the growth environment. For example, a portion of the growth environment can refer to the unit length, the unit width, the unit height, the unit body diagonal, the unit face diagonal, the unit perimeter, the unit radius, the unit circumference, the unit surface area, the unit cross section, or the unit volume of the growth environment.

The geometry of the growth environment can be customized to support mycelium growth at several spatial scales. In some embodiments, the volume of the growth environment can fall within a range of between about at least 0.1 ft³ and/or less than or equal to about 500,000 ft³, or can fall within a range between about at least 1.0 ft³ and/or less than or equal to 250,000 ft³. In some yet further embodiments, the volume of the growth environment can be about 0.1 ft³, 0.2 ft³, 0.3 ft³, 0.4 ft³, 0.5 ft³, 0.6 ft³, 0.7 ft³, 0.8 ft³, 0.9 ft³, 1.0 ft³, or any range therebetween. In some yet further embodiments, the volume of the growth environment can be about 250,000 ft³, 300,000 ft³, 400,000 ft³, 500,000 ft³, or any range therebetween.

“Aerial mycelium” as used herein refers to mycelium obtained from extra-particle aerial mycelial growth, and which is substantially free of growth matrix.

“Extra-particle mycelial growth” (EPM) as used herein refers to mycelial growth, which can be either appressed or aerial.

“Extra-particle aerial mycelial growth” as used herein refers to a distinct mycelial growth that occurs away from and outward from the surface of a growth matrix. Extra-particle aerial mycelial growth can exhibit negative gravitropism. In a geometrically unrestricted scenario, extra-particle aerial mycelial growth could be described as being positively gravitropic, or neutrally gravitropic, aerial, and radial in which growth will expand in all directions from its point source. In some embodiments, external forces, such as airflow, can be applied towards (e.g., approximately perpendicular to the growth environment floor) the growth substrate, and in some embodiments, through the growth substrate, for example, to create downward aerial mycelium growth in the direction of gravity. Alternatively, airflow can be applied across the growth substrate in a manner parallel or horizontal to the growth substrate surface.

“Positive gravitropism” as used herein refers to growth that preferentially occurs in the direction of gravity.

“Negative gravitropism” as used herein refers to mycelial growth that preferentially occurs in the direction away from gravity. As disclosed herein, extra-particle aerial mycelial growth can exhibit negative gravitropism. Without being bound by any particular theory, this may be attributable at least in part to the geometric restriction of the growth format, wherein an uncovered tool having a bottom and side walls contains a growth matrix. With such geometric restriction, growth will primarily occur along the unrestricted dimension(s), which in the scenario is primarily vertically (negatively gravitropic).

Aerial mycelia of the present disclosure can be grown in a matter of weeks or days. This feature is of practical value in the production of food ingredient or food product, where time and efficiency are at a premium. Accordingly, the presently disclosed method of making an aerial mycelium comprises incubating a growth matrix in a growth environment for an incubation time period of up to about 3 weeks. In some embodiments, the incubation time period can be within a range of about 4 days to about 17 days. In some further embodiments, the incubation time period can be within a range of about 7 days to about 16 days, within a range of about 8 days to about 15 days, within a range of about 9 days to about 15 days, within a range of about 9 days to about 14 days, within a range of about 8 to about 14 days, within a range of about 7 to about 13 days, or within a range of about 7 to about 10 days. In some more particular embodiments, the incubation time period can be about 7 days, about 8 days, about 9 days, about 10 days, about 11 days, about 12 days, about 13 days, about 14 days, about 15 days or about 16 days, or any range therebetween.

Advantageously, incubating a growth matrix comprising a colonized substrate (wherein said colonized substrate comprises a growth medium previously colonized with mycelium of a fungus) in a growth environment of the present disclosure can result in earlier expression of aerial mycelial tissue compared to incubation of a growth matrix comprising substantially the same or a similar growth medium and a fungal inoculum, wherein the fungal inoculum contains a fungus. Accordingly, a method of making an aerial mycelium of the present disclosure can comprise incubating a growth matrix comprising a colonized substrate (wherein said colonized substrate comprises a growth medium previously colonized with mycelium of a fungus) in a growth environment for an incubation time period, and producing extra-particle aerial mycelial growth therefrom, wherein the incubation time period is at least about 1 day, at least about 2 days, at least about 3 days, or at least about 4 days less than the incubation time period for producing extra-particle aerial mycelial growth from a growth matrix comprising a growth medium and a fungal inoculum, wherein the fungal inoculum comprises a fungus.

In some other embodiments, the incubation time period ends no later than when a visible fruiting body forms. In a non-limiting example, the incubation time period can end prior to a karyogamy or meiosis phase of the fungal reproductive cycle. In some other embodiments, the incubation time period ends when a visible fruiting body forms. As disclosed herein, aerial mycelia of the present disclosure can be prepared without the formation of a visible fruiting body, thus, in some embodiments, an incubation time period can end without regard to the formation of a visible fruiting body. Trial incubation runs can be used to inform the period of time in the growth environment during which sufficient extra-particle aerial mycelial growth product occurs (e.g., aerial mycelial growth of a predetermined thickness) without the formation of visible fruiting bodies.

In some embodiments, the method of making an aerial mycelium of the present disclosure can comprise introducing aqueous mist into the growth environment throughout the incubation time period.

In some embodiments, aerial mycelia can be prepared by exposing a growth matrix to aqueous mist throughout a portion of the incubation time period (e.g., by introducing mist into the growth environment throughout a portion of the incubation time period). Applicant has measured vertical expansion kinetics of mycelia over the course of an entire incubation period and has characterized the kinetics as having a primary myceliation phase and a vertical expansion phase. The primary myceliation phase included days 1 to 3 of the incubation time period. Introducing aqueous mist throughout a portion of the incubation time period (wherein the portion included the vertical expansion phase), and not introducing aqueous mist on days 1 to 3 of the incubation time period was sufficient to produce aerial mycelium having substantially similar characteristics to aerial mycelia obtained by depositing mist throughout the entire incubation period.

The desired airborne mist concentration value, and/or the control of the airborne mist concentration level in response to the mist concentration value, for improved growth may be different during different phases of the growing cycle (including zero). Further, the desired airborne mist concentration value, and/or the control of the airborne mist concentration level in response to the mist concentration value, for improved growth may also be different based on the organism generating the aerial mycelia. Some aspects of the present disclosure provide for a method of making an aerial mycelium comprising exposing a growth matrix to a growth environment comprising aqueous mist throughout the incubation time period (e.g., by introducing aqueous mist into the growth environment throughout the incubation time period, i.e., throughout the entire incubation time period). In other aspects, the present disclosure provides for a method of making an aerial mycelium comprising exposing a growth matrix to aqueous mist throughout a portion of the incubation time period (e.g., by introducing aqueous mist into the growth environment throughout a portion of the incubation time period). In some embodiments, a portion of the incubation time period can comprise a vertical expansion phase. In some further embodiments, a portion of the incubation time period can further comprise at least a portion of a primary myceliation phase. In some other embodiments, a portion of the incubation time period can exclude a primary myceliation phase. In yet some other embodiments, a portion of the incubation time period can comprise a vertical expansion phase. Accordingly, in some aspects, introducing aqueous mist into a growth environment throughout a portion of an incubation time period can comprise introducing aqueous mist into the growth environment throughout a vertical expansion phase. In some embodiments, introducing aqueous mist into the growth environment throughout a portion of the incubation time period can comprise introducing aqueous mist into the growth environment throughout a vertical expansion phase and can exclude introducing aqueous mist during the primary myceliation phase. In some embodiments, the portion of the incubation time period can terminate at the end of a vertical expansion phase or can terminate at the end of an incubation time period.

In some other aspects, a portion of an incubation time period can begin during a first day, a second day, a third day or a fourth day of the incubation time period. Accordingly, in some aspects, introducing aqueous mist into a growth environment throughout a portion of an incubation time period can comprise introducing aqueous mist into the growth environment during a first, a second, a third or a fourth day of the incubation time period. In some embodiments, the portion of the incubation time period can terminate at the end of a vertical expansion phase or can terminate at the end of an incubation time period.

“Near-Infrared” as used herein refers to electromagnetic radiation comprising a wavelength greater than or equal to 780 nm and less than or equal to 2,800 nm.

### DISCUSSION OF THE FIGURES

The following discussion presents detailed descriptions of the several embodiments of apparatus, systems, and methods for growing mycelium, for example through detection of an airborne mist concentration within a growth environment, as shown in the figures. These embodiments are not intended to be limiting, and modifications, variations, combinations, etc., are possible and within the scope of this disclosure.

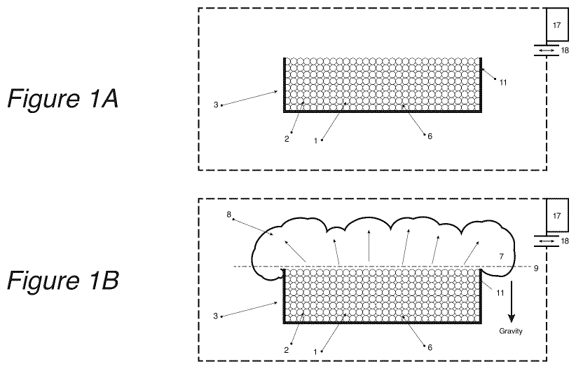

FIG. 1A illustrates an embodiment of a growth matrix 3 suitable to support extra-particle mycelial growth, such as extra-particle aerial mycelial growth. The growth matrix 3 is shown as circles. In some embodiments, the growth matrix 3 can be contained within a tray 11 with a bottom and side walls as shown. The growth matrix 3 can include a growth medium and a fungus. For example, the growth matrix 3 can comprise growth media 2, substrate 1, and colonized (or pre-colonized) substrate 6, to support growth therefrom. The growth matrix 3 can be contained within a growth environment, shown in dashed lines. One or more environmental conditions can be controlled within the growth environment, for example, to affect the growth from the growth matrix for desirable results. For example, a processor 17 can be provided to control oxygen (O₂) content, carbon dioxide content (CO₂), temperature, humidity, misting, and/or other environmental conditions, to, from, and/or within the growth environment, for example, through an interface 18.

In some embodiments, the growth matrix 3 is implemented without tray 11 (e.g., on another growth support structure, such as a planar support structure without side walls, such as a mycological growth web).

FIG. 1B illustrates an embodiment of extra-particle aerial mycelial growth 8 from the growth matrix 3 of FIG. 1A. For example, the growth can occur when the growth matrix 3 from FIG. 1A is incubated or otherwise processed within a growth environment under growth conditions suitable for the desired properties of the extra-particle aerial mycelium growth 8 in FIG. 1B. For example, in some embodiments, the environmental condition(s) within the growth environment can be controlled to induce substantially homogeneous extra-particle aerial mycelial growth from the growth matrix.

The extra-particle aerial mycelium growth can extend away from and outward from a surface of the growth matrix to form an aerial mycelium 7 as shown. Appropriate growth conditions of the growth matrix 3 in FIG. 1A result in extra-particle aerial mycelium growth initiating across the exposed surface. Next, extra-particle aerial mycelium growth continues to expand forming a volume of extra-particle mycelial growth 8 as shown in FIG. 1B. The volume of growth can be contiguous. The extra-particle aerial mycelium growth 8 can be grown to various heights. In some embodiments, the growth is about 3-4 inches high above the growth matrix 3. This can be achieved, for example, in up to two weeks of growth. It will be understood that although the extra-particle aerial mycelium growth has some amount of irregularity to its upper surface topology as shown, the drawings are not to scale, and the top surface can be relatively flat. In some embodiments, the top surface may be processed in additional steps, to improve the surface flatness, uniformity, and/or other desirable features. A separation zone 9 (dot-dashed line) can be defined as a zone where the extra-particle aerial mycelium growth 8 can be divided and detached from the growth matrix 3.

In some embodiments, the growth can be implemented on a mycological growth web, for example, without the tray 11 shown. The growth web can include the growth matrix and the extra-particle aerial mycelial growth (e.g., without a tray 11). The growth web can include any suitable support structure to support the growth matrix 3 and the extra-particle aerial mycelium growth 8, such as a growing net. The web can be a standard size, such as a 63″W×38′L, 63″W×98′L or any of many other web configurations. Other sizes can be implemented, including lengths up to 90, 100 feet, or more. The growing net can comprise one or more layers of a perforated or nonperforated material, or combinations thereof, such as a plastic, nylon (e.g., nylon weave), or any other flexible, suitable material or multiple layers of material for growing extra-particle aerial mycelium growth 8 from a growth matrix 3. The web can extend in length from right to left in the orientation shown in FIG. 1B.

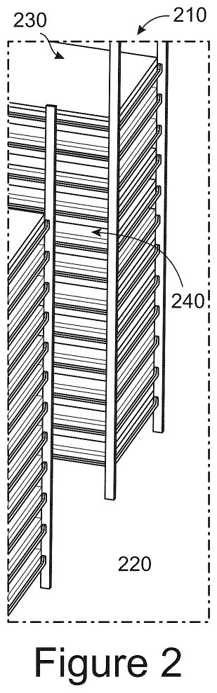

FIG. 2 illustrates an embodiment of an isometric view of a system 210 for cultivating aerial mycelia within a growth environment 220. The growth environment 220 can be configured to grow mycelium from a growth matrix positioned therewithin. For example, system 210 and growth environment 220 can be implemented to facilitate the mycelial growth described above with reference to FIGS. 1A-1B, and can include similar components and functionality.

The growth environment 220 can include one or more shelves 240 (e.g., vertically configured shelves), on which a growth matrix can be positioned, and from which extra-particle mycelial growth can extend. The growth matrix can be positioned directly on a shelf, or with an intervening growth support structure, such as a growth web. One or more racks 230, such as the two racks shown, can include a plurality of the shelves 240 (e.g., stacked vertically), positioned within the growth environment 220. The environment can include various numbers of racks, which can include various numbers of shelves 240, and can be of various dimensions. For example, two or more racks 230 can form two corresponding sets of shelves 240, wherein each shelf in a first set on a first rack can be positioned at approximately the same height as a corresponding shelf in the other set of shelves on the other rack. A shelf can be sized and configured to support a web or other growth support structure, such as those described above with reference to FIGS. 1A and 1B, or other growth support structures. In some embodiments, the racks include 12 shelves, and are 12-16 feet high. The shelves 240 can move vertically to compensate for spacing shelves 240 at different heights. The shelves 240 can be spaced apart from each other (e.g., vertically) a spacing height H that is sufficient to allow for the height of the desired extra-particle aerial mycelial growth combined with the height of the underlying growth matrix, sufficient clearance for airflow, mist flow, processing, and handling. It will be understood that air, air content and airflow as used herein can refer to various compositions of various gases, and flow of those gases suitable for the products and methods described herein, and should not otherwise be limited to a particular composition or ratio of gases. Additionally, some embodiments herein that are described as being implemented with respect to “water” may be similarly implemented with other liquids.

The spacing height H is defined as the distance between the same two corresponding points on two adjacent shelves 240. For example, the spacing height can be defined as the distance from the top surface of a first lower shelf to the corresponding top surface of an adjacent upper shelf. In some embodiments, the spacing height can be in a range between about 200 mm to about 530 mm, or between about 225 mm to about 490 mm, or between about 250 mm to about 450 mm. In some embodiments, the spacing height can be less than 530 mm, less than 490 mm, or less than 450 mm. In some embodiments, the spacing height can be about 350 mm. These spacing heights can be advantageous because the nature of the mycelial growth herein requires less spacing, and thus can allow for an increased number of shelves and higher output than conventional mushroom cultivation.

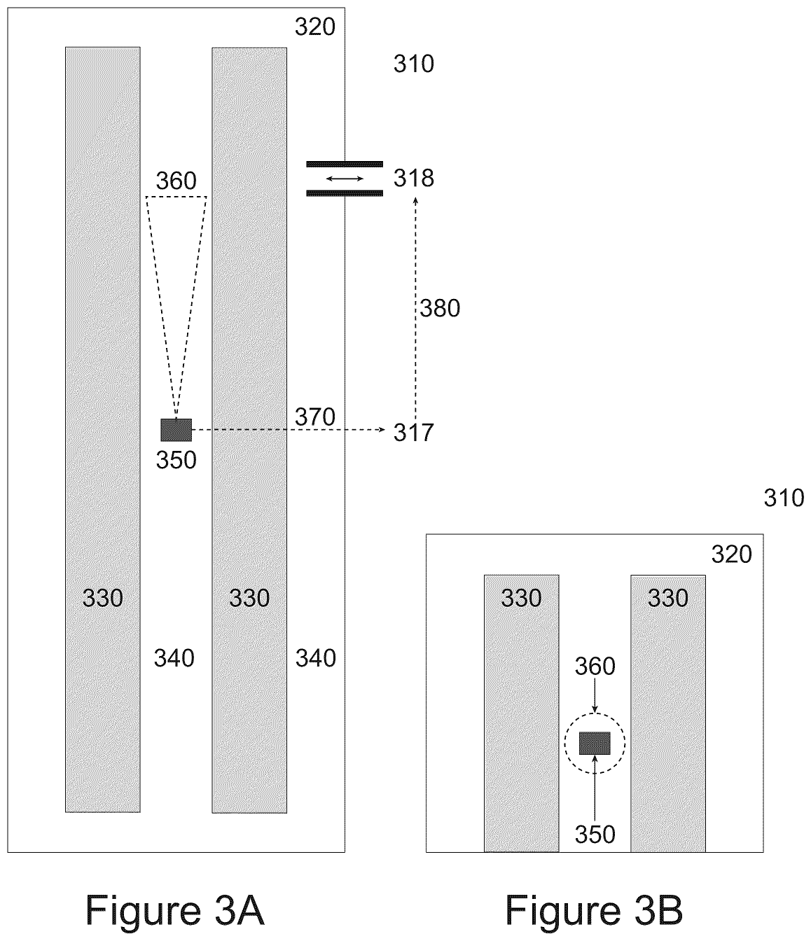

In some embodiments, the volume of the growth environment can fall within a range of between about at least 0.1 ft³ and/or less than or equal to about 500,000 ft³, or can fall within a range between about at least 1.0 ft³ and/or less than or equal to 250,000 ft³. [0083] FIGS. 3A and 3B illustrate a top and front view, respectively, of an embodiment of a system 310 configured to electronically detect a mist concentration value within a growth environment 320. The growth environment 320 can include one or more shelves 330 or shelving units on one or more racks 340. System 310 and its components can be substantially similar as and can function substantially similar to system 210 and its components, as described above with reference to FIG. 2.

The system 310 can include one or more sensor(s) of any configuration suitable to electronically detect a mist concentration value, and in some embodiments, an airborne mist concentration value within the growth environment 320, such as a sensor 350. In some embodiments, such system can provide such sensing without measuring relative humidity by any sensor 350. The sensor 350 can electronically detect and generate a signal 370 that is indicative of a mist concentration value (e.g., airborne mist concentration value) within the growth environment 320. The sensor(s) 350 can comprise various types of mist concentration sensor(s) 350, implementing various types of technologies, such as visual, optical, near-infrared or infrared, laser, chemical, ultrasonic, radar, LED, and/or other types of sensor(s) and technologie(s). For example, a camera and/or a modified camera can capture images or continuous video of the interior of a growth chamber. These images can be analyzed to determine the concentration of mist present in the growth chamber. In some embodiments, the technology could determine the amount of airborne droplets based on the chemical signature of H₂O or ingredients/elements that are loaded within the H₂O mist. Such elements would need to be inert essentially (or nutritive), such that they do not have any meaningful negative impact on mycelium growth or life cycle development, or for that matter, do not pose any negative impact during human consumption (for food applications) or from skin absorption in the case of textile applications (e.g., garments/clothing). For example, an inert dye or other additive could be placed within the liquid (e.g., water) going into the system and a spectral sensor could be operating to detect the liquid droplets or dye, rather than a visual sensing of fog itself. In some embodiments, the signal 370 is transmitted and detected by various means, for example, using a reflector, a light source, an infrared light source, a laser diode, and/or other means. For example, attenuation in the frequency (Hz), intensity (W/m2) or wavelength (nm) of light emitted from or received by the sensor 350 can be measured relative to a baseline value to determine the amount of airborne droplets suspended in an airmass. In some further embodiments, the rate of mist deposition can be determined by measuring and comparing, for example, light attenuation values from two or more time points.

In some embodiments, the concentration of airborne mist can be measured within a one-dimensional, two-dimensional or three-dimensional section of an airmass. In one embodiment, the concentration of airborne mist can be measured across a one-dimensional vector that transects an airmass within the growth environment 320. For example, a sensor 350 can measure the concentration of airborne mist in a one-dimensional vector of a length of 0.01 in, 0.1 in, 1 in, 10 in, or any range therebetween. In some further embodiments, the concentration of airborne mist can be measured within a two-dimensions area of airmass within the growth environment 320. For example, a sensor 350 can measure the concentration of airborne mist in the growth environment 320 with an area of 0.0001 ft², 0.01 ft², 1.0 ft², 100 ft² or any range therebetween. In a further embodiment, a sensor 350 can measure the concentration of airborne mist in a three-dimensional volume of air within the growth environment 320. For example, a sensor 350 can measure the concentration of airborne mist within the growth environment with a volume of 1×10⁻⁶ ft³, 0.0001 ft3, 1.0 ft³, 1,000 ft³, or any range therebetween. In some further embodiments, the concentration of airborne mist in the growth environment 320 is measured at a distance from a sensor 350 that can range from 0.01 in to 500 ft.

In further embodiments, the sensor 350 can be calibrated in an environment absent airborne mist. The sensor 350 can be calibrated by placing the sensor 350 in a stationary position in front of a flat surface. Mist concentration data can be recorded as the sensor 350 is operated. These data can then be aggregated by, for example, averaging mist concentration data from multiple tests over desired periods of time to obtain a base line mist concentration value. The baseline value can then be used to calibrate and standardize sensor 350 sensitivity within the grown environment 320.

In some embodiments, the density of airborne mist will be greater than the density of air or water vapor, so the bulk density of the mixture may vary based on the concentration of liquid (e.g., water) droplets in the airborne mist. This change in mass may impact sonar, ultrasonics, or other vibration-based detection methods, for which the processor and its control algorithms can compensate.

The sensor 350 can be configured to electronically communicate the first signal 370 to a processor 317. The processor 317 can be configured to receive the signal 370. In response to the signal 370, processor 317 can be configured to control (e.g., be programmed to control) various environmental conditions to, from, and/or within the growth environment 320, for example, through an interface 318. In some embodiments, in response to the signal 370, the processor 317 can be configured to control oxygen (O₂) content, carbon dioxide content (CO₂), temperature, humidity, and/or other environmental conditions, to, from, and/or within the growth environment 320.

The processor 317 can be configured to control (e.g., increase, decrease, or maintain without change) an environmental condition within growth environment 320 to a desired environmental condition value. For example, the processor 317 can be configured to increase an environmental condition, such as the amount of airborne mist, within the growth environment 320, when the airborne mist concentration value is below a first desired value. The processor 317 can be configured to decrease an environmental condition, such as the amount of airborne mist, within the growth environment 320, when the airborne mist concentration is above a second desired value. For example, the amount of airborne mist may be decreased within the growth environment 320 if there is too much airborne mist for a particular stage in a growth cycle, for example, if a stage of the growth cycle has been reached where such mist offers no or little advantage. In one embodiment, the amount of airborne mist may be decreased within the growth environment 320 by pausing and/or stopping mist input into the growth environment 320 and allowing mist droplets to settle from the air due to gravity. In one embodiment, the mist may be decreased when various non-desirable physical features are observed on the aerial mycelium either through human or machine assisted monitoring, and the level of mist in accordance with the sensor is perceived to be at an excessive level.

The first environmental condition value and the second desired environmental condition value can be the same or different value relative to each other. In some embodiments, processor 317 can be configured to control by taking no action. A growth environment condition can be maintained by preventing any substantial change in an environmental condition value or window of values between two or more time points. In this way, an environmental condition can be maintained when it is approximately equal to or at a desired environmental condition value, or at or between a first desired value (e.g., lower threshold) and a second desired value (e.g., upper threshold).

In order to provide such control, the processor 317 can generate a processor output 380 to control another component of system 310, such as interface 318. Interface 318 can be configured to increase, decrease, or maintain without change an environmental condition within growth environment 320 in response to the output 380 received from processor 317. For example, the processor 317 can be configured to control (e.g., change, or maintain constant) an airborne mist concentration level or other environmental conditions within growth environment 320, in response to signal 370. The interface 318 can include one or more inputs and/or outputs to adjust one or more environmental conditions. For example, the interface 318 can increase the amount of airborne mist within the growth environment 320, for example, by introducing aqueous mist into the growth environment 320 through one or more growth environment inlet(s). It will be understood that the inlet(s) can be positioned anywhere within, or associated with, the growth environment 320 suitable for the desired misting results, such as proximate to an outer portion (e.g., sidewall) of the growth environment 320 as shown, e.g., within an air handling unit associated with the growth environment 320, or closer to the growth matrix (e.g., closer to or mounted on the growing shelves or racks). The interface 318 can decrease the amount of airborne mist within the growth environment 320, for example, by removing aqueous mist from the growth environment 320 through a growth environment outlet. Alternatively, the amount of airborne mist within the growth environment 320 can be decreased, for example, by pausing and/or stopping mist entry into the growth chamber through the interface and allowing airborne mist droplets to settle from the air column due to gravity. The amount of airborne mist within the growth environment 320 can also be adjusted, for example, by managing the water carrying capacity of the air (e.g., warmer air at the inlet), introducing temperature gradients (e.g., warm air/cooler air and surfaces), using air pressure gradients to localize temperature air temperature change (e.g., expanding air cools, air velocity creates pressure gradients), and/or other ways.

The inlets and outlets can comprise the same component, or can comprise different, separate components. In some embodiments, the sensor 350 is located in the center of the growth environment 320, but it will be understood by one skilled in the art that the sensor 350 can be placed anywhere within the growth environment 320 suitable to provide the functionality described herein. In some embodiments, the sensor 350 is placed in the center of the growth environment 320 to allow broad measures of the growth environment 320 as a whole. In some further embodiments, the sensor 350 can be placed in non-central locations in the growth environment 320 to determine growth environment 320 performance in specific geographic areas. In some further embodiments, the sensor can be placed outside of the growth environment 320. For example, the sensor can be configured to detect a mist concentration value through an exterior wall or window of the growth environment 320.

In some embodiments, the sensor 350 can generate a first signal 360 that is indicative of airborne mist concentration. In some embodiments, the signal 360 is transmitted substantially parallel to the sensor 350, but it will be understood by one skilled in the relevant art that the signal 360 can be transmitted at any angle with respect to the sensor 350. In some further embodiments, the signal 360 is transmitted through a conical volume of empty space within the growth environment 320. In some embodiments, the radius and height of the conical volume are a function of signal 360 intensity. In some further embodiments, the signal can be transmitted through a volume of empty space that comprises a portion of the total volume of the growth environment 320 or the entirety of the total volume of the growth environment 320a.

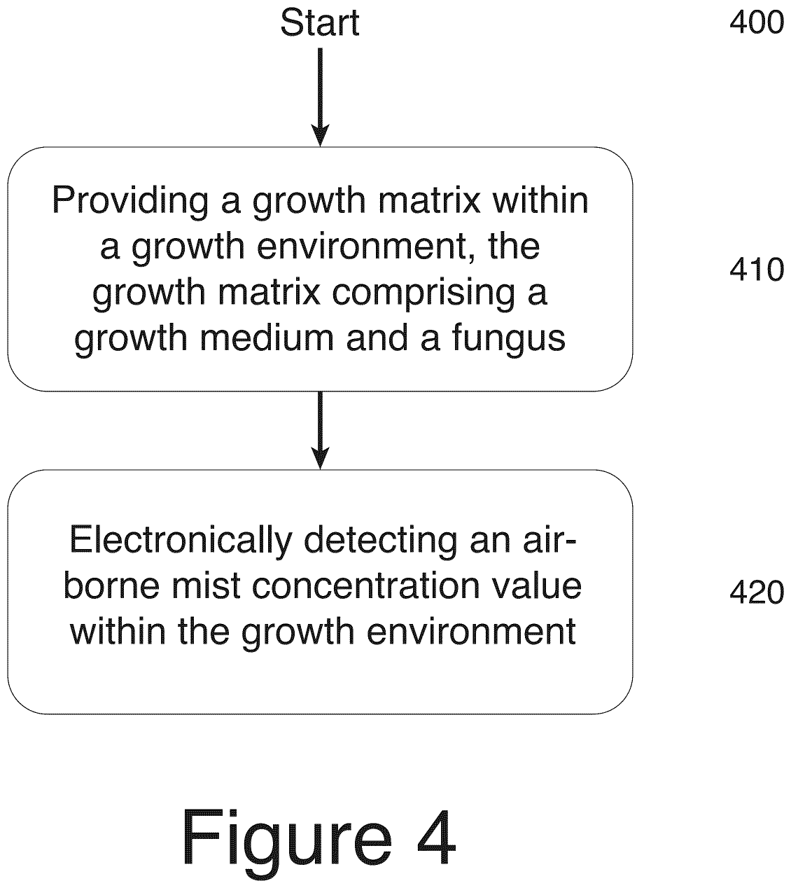

FIG. 4 illustrates a flow diagram of an embodiment of a method 400 for growing mycelium. The method 400 can include a step 410 of providing a growth matrix within a growth environment. The growth matrix can include a growth medium and a fungus. The method 400 can include a step 420 of electronically detecting an airborne mist concentration value within the growth environment. Step 420 can further include controlling an airborne mist concentration level within the growth environment in response to an airborne mist concentration value. Controlling can include at least one of increasing, decreasing or maintaining an airborne mist concentration level in a growth chamber when the airborne mist concentration value is below, above and/or at/between a first desired value and/or a second desired value. For example, increasing the amount of airborne mist within the growth environment can comprise introducing mist into the growth environment, for example, through a growth environment inlet. Decreasing the amount of airborne mist within the growth environment can comprise removing mist from the growth environment, for example, through a growth environment outlet. In some embodiments, decreasing the amount of airborne mist within the growth environment can comprise pausing, reducing, and/or stopping the airborne mist introduction into the growth environment, for example, to allow airborne mist droplets to settle out of the air due to gravity, and/or be consumed by the mycelium growth In some embodiments, maintaining the airborne mist concentration level in a growth chamber can comprise introducing mist into the growth environment to maintain an approximately steady-state mist concentration level. For example, maintaining the airborne mist concentration level can comprise introducing an amount of airborne mist into the growth environment at a rate that is approximately equal to, and thus compensates for, the rate of loss of airborne mist concentration in the chamber, for example, due to the settling of airborne mist droplets due to gravity, and/or being consumed by the mycelium growth, or other reasons.

In some embodiments, step 420 can include comparing the first desired value to a second desired value, wherein the first desired value and the second desired value are different values relative to each other. Step 420 can include electronically detecting an airborne mist concentration value with a sensor located within the growth environment. In some embodiments two or more sensors can be employed. Step 420 can include the use of an infrared fog sensor to detect airborne mist concentrations in the growth chamber.

The method 400 can be implemented with the various systems, methods, components and apparatus described herein with respect to FIGS. 1A-3B, or in other applications. For example, method 400 can be implemented with the growth matrix 3 in FIG. 1A, to grow the extra-particle aerial mycelium growth 8 in FIG. 1B. The method 400 can be implemented with the sensor 350 to detect an airborne mist concentration value within the growth environment 320 as described and shown in FIG. 3.

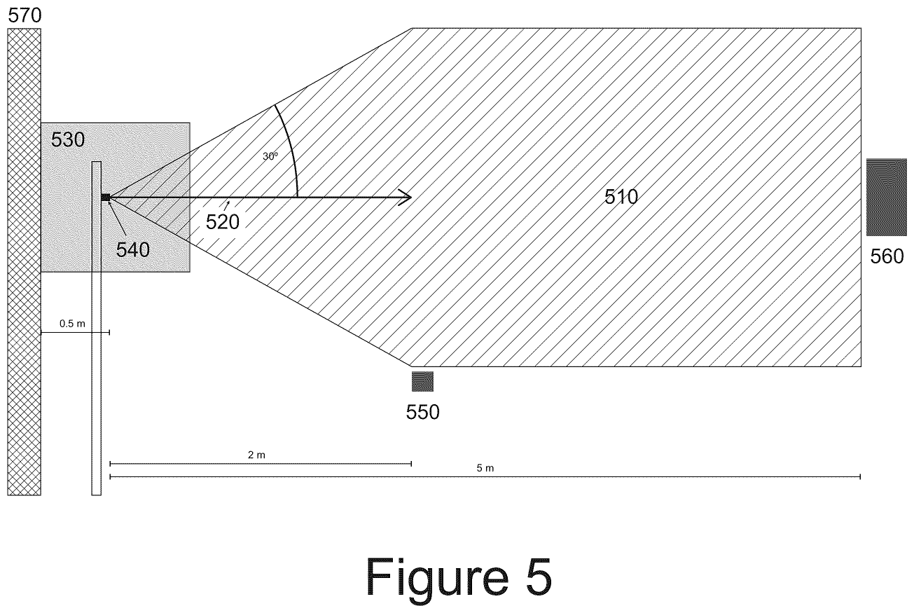

FIG. 5 illustrates an exemplary lateral view of a mounted sensor 540. In some preferred embodiments, the sensor 540 is mounted at least 0.5 m from a solid surface, e.g., a wall of a growth chamber of a growth environment. Friction causes pockets of air to poorly circulate when adjacent to solid surfaces, and as a result, the sensor 540 is depicted 0.5 m from a vertical surface 570 as an exemplary embodiment to improve sensor 540 exposure to circulating air. In some embodiments, the sensor 540 can be any distance greater than 0.5 m from a solid surface. In some further embodiments, a volume free of any objects 530 surrounds the sensor 540.

In some embodiments, the sensor 540 transmits a signal 520 that is substantially parallel to the sensor 520, although it will be understood by one skilled in the art that the signal 520 can be transmitted at any angle with respect to the sensor 540. In some embodiments the signal 540 is transmitted into a volume free of any solid objects 510. In some embodiments, the volume free of any solid objects 510 extends conically to a distance of at least 2 m. FIG. 5 illustrates an embodiment of a lateral cross-section view of a conic volume free of any solid objects 510, where the vertex is 30 degrees from the signal 520. As an example, FIG. 5 illustrates a solid object 550 present at a distance from the sensor 540, shown to be 2 m away from the sensor 540 at an angle of 30 degrees below the signal 520. In some further embodiments, the volume free of any solid objects 510 extends cylindrically to a distance of at least 5 m. For example, FIG. 5 illustrates how a solid object 550 that is 2 m from the sensor 540 causes the transmission of the signal 520 to change from conic to cylindric. After 2 m, the signal 520 is transmitted away from the sensor 540 at an angle of 0 degrees until the signal is obstructed by a solid object 560, for example, 5 m away from the sensor 540.

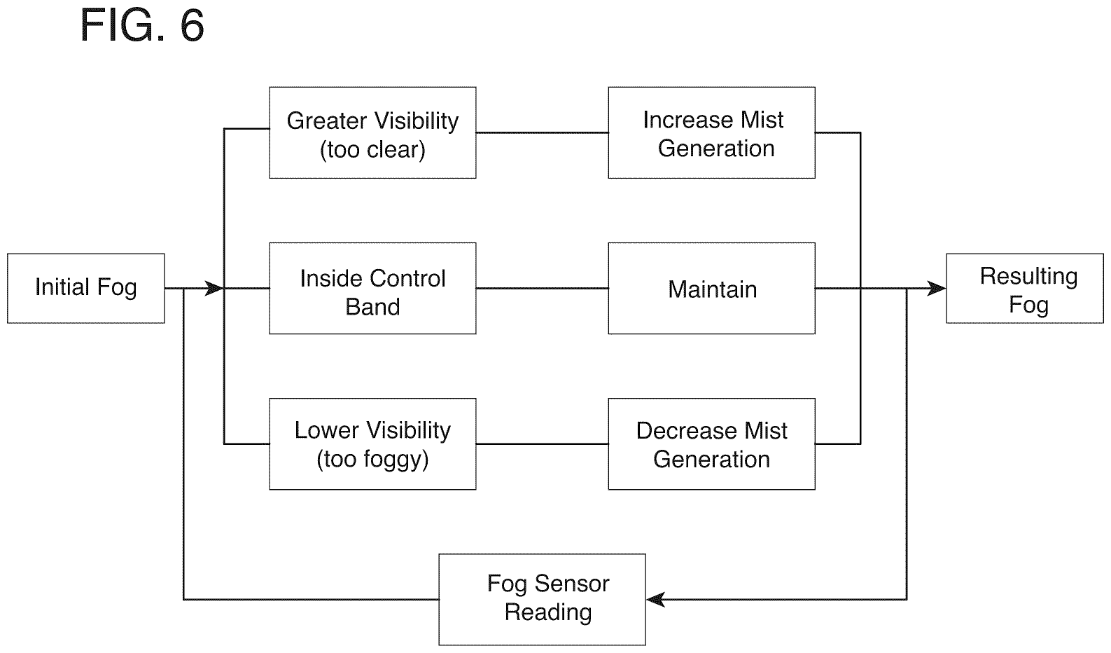

FIG. 6 illustrates a flow diagram of an embodiment of control logic for detecting and controlling airborne mist within a growth environment. The functional aspects of the control logic shown can be implemented with the sensor(s) and processor(s) described herein. Any reference to the term “fog” can be replaced with “airborne mist” as described herein, and any reference to “visibility” can be supplemented or substituted with other types of sensors and detection described herein. Embodiments herein can include more complex control methodologies, and/or can be used alone or in combination with other parameters and/or environmental conditions, such as timing and/or relative humidity.

Additional Context for Light Based Methods for Mist Detection: The following provides a contemplated variety of light-based sensor methods for mist detection which rely on the basic principle that an aqueous particle will interact with and diffuse or reflect incoming light.

In an embodiment of a sensor, an emitter is located next to a detector, both facing in the same direction. Light is emitted and absent any fog to interact with, is not detected. As the volume being measured is filled with fog, more and more of the light emitted is reflected back to the sensor and picked up by the detector. This is scattered or backscattered detection.

In order not to be confused by any other sources of light in an environment, certain techniques are contemplated to be used. First, a very specific wavelength may be emitted and detected. Second, the emission may not be continuous but presented as a phased burst. Detection may then separate out the light which is universally present in the environment from the light which is present only during the phased burst in order to get an accurate signal independent of other sources. One downside to a backscatter approach is that it requires an operating volume in front of the sensor which is free from obstructions except for the fog.

An alternate arrangement of the emitter and detector contemplated, may be to have them facing each other across a certain gap, but slightly misaligned by a desired angle. The misalignment would serve such that when the emitter is on but there is no fog, no light should be detected by the detector (the light being focused in a narrow beam which misses the detector). As fog is introduced, the particles scatter the light from the emitter, increasing the amount of light which reaches the detector. The same techniques for signal to noise optimization as described above can be used here. This method may be considered a form of forward scattering or “off-axis.” One advantage of this arrangement is that the volume being measured for fog is only that which is situated in between the sensor and emitter, so a much smaller overall sensing area can be obtained, which allows for sensing within a very specific or small section of a larger growth environment.

A third combination of emitter and detector contemplated is to have them facing each other and directly aligned on an axis. In this arrangement, the emitter can be a laser (i.e., a highly concentrated beam) and the detector can be looking for breaks or reductions in the signal as opposed to deflected light. A break in the signal can indicate that a particle has crossed into the beam. Information about the break durations and frequency can be used to infer particle sizes if the velocity across the beam is known. This style of sensor may be considered a “break-beam”-style sensor.

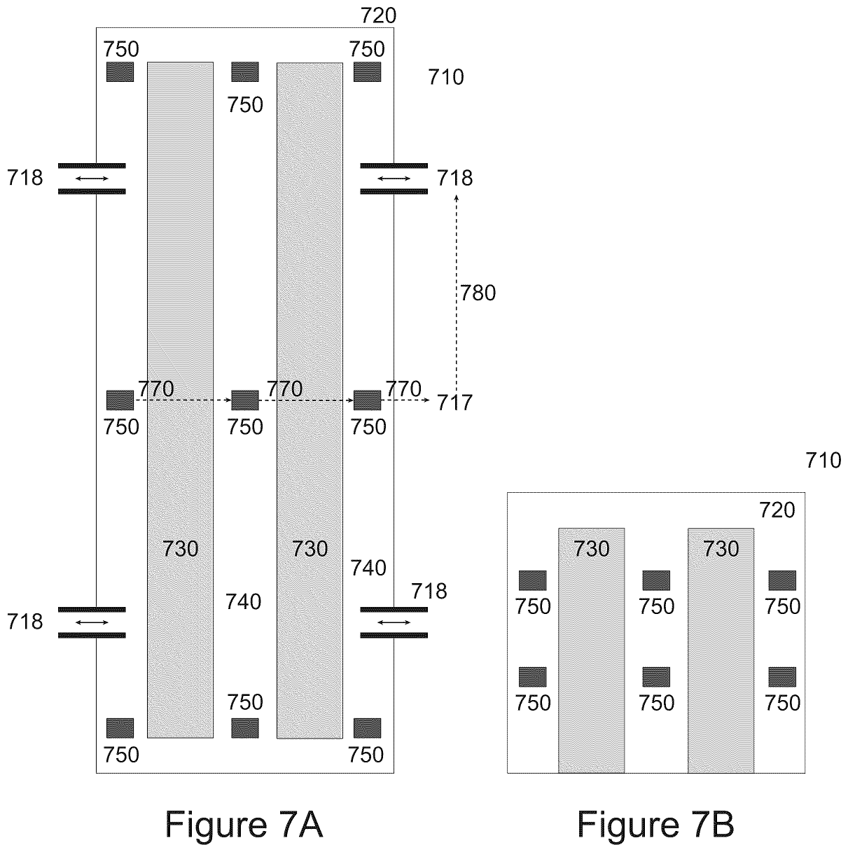

FIGS. 7A and 7B illustrate a top and front view, respectively, of an embodiment of a system 710 configured to electronically detect a mist concentration value within individual portions of the growth environment 720. The growth environment 720 can include one or more shelves 730 or shelving units on one or more racks 740. System 710 and its components can be substantially similar as and can function substantially similar to system 210 and 310, and their components, as described above with reference to FIG. 2 and FIGS. 3A and 3B, respectively. A difference is that the system 710 can include additional sensor(s), and other components and/or functionality as follows:

The system 710 can include one or more sensor(s) of any configuration suitable to electronically detect a mist concentration value, and in some embodiments, an airborne mist concentration value within the growth environment 720, such as multiple sensors 750. In some embodiments, such system can provide such sensing without measuring relative humidity by any sensor 750. The sensors 750 can electronically detect and generate a signal 770 that is indicative of a mist concentration value (e.g., airborne mist concentration value) within the growth environment 720. The sensor(s) 750 can comprise various types of mist concentration sensor(s) 750, implementing various types of technologies, such as visual, optical, near-infrared, infrared, laser, chemical, ultrasonic, radar, LED and/or other types of sensor(s) and technologie(s). For example, multiple sensors can be placed throughout the growth environment 720. These sensors 750 can continuously monitor a portion of the growth environment 720 to provide mist concentration values with finer granularity and with respect to the portion of the growth environment 720 in which they are located. In some embodiments, the sensors 750 can comprise a near-infrared or infrared-emitting LED and a phototransistor. The phototransistor can measure the intensity of near-infrared or infrared light reflected by airborne mist. The greater the intensity of reflected near-infrared light, the greater the concentration of airborne mist in the growth environment 720. In some embodiments, the sensors 750 can measure airborne mist concentration levels within a range of between about at least 0.1 in from the sensor 750 and/or less than or equal to about 500 ft from the sensor 750, or 1.0 in from the sensor 750 and/or less than or equal to 100 ft from the sensor 750. In some yet further embodiments, airborne mist concentration levels can be measured outwards from a sensor 750 within about 0.1 in, 0.05 in, 1 in, 2 in, 3 in, 4 in, 5 in, 6 in, 7 in, 8 in, 9 in, 10 in, 12 in, 13 in, 14 in, or 15 in, 2 ft, 3 ft, 4 ft, 5 ft, 10 ft, 15 ft, 20 ft, 25 ft, 50 ft, 75 ft, 100 ft, 125 ft, 150 ft, 175 ft, 200 ft, 225 ft, 250 ft, 275 ft, 300 ft, 325 ft, 350 ft or 400 ft, or any range therebetween. In some further embodiments, a heating element can be combined or juxtaposed with a sensor 750 to prevent the formation of condensation on the sensor or LED. Attenuation in the frequency (Hz), intensity (W/m2) or wavelength (nm) of light emitted from or received by the sensor 750 can be measured relative to a baseline value to determine the amount of airborne droplets suspended in an airmass. In some further embodiments, the rate of mist deposition can be determined by measuring and comparing, for example, light attenuation values from two or more time points. In some further embodiments, the sensors 750 can be calibrated in an environment absent airborne mist.

In some further embodiments, the sensor 750 can be calibrated in an environment absent airborne mist. The sensor 750 can be calibrated by placing the sensor 750 in a stationary position in front of a flat surface. Airborne mist concentration data can be recorded as the sensor 750 is operated. These data can then be aggregated by, for example, averaging mist concentration data from multiple tests over desired periods of time to obtain a base line mist concentration value. The baseline value can then be used to calibrate and standardize sensor 750 sensitivity within the grown environment 720.

In some embodiments, the density of airborne mist will be greater than the density of air or water vapor, so the bulk density of the mixture may vary based on the concentration of liquid (e.g., water) droplets in the airborne mist. This change in mass may impact sonar, ultrasonics, or other vibration-based detection methods, for which the processor and its control algorithms can compensate.

Each sensor 750 can be configured to electronically communicate the first signal to a processor 717. The processor 717 can be configured to receive the signal 770. In response to the signal 770, processor 717 can be configured to control (e.g., be programmed to control) various environmental conditions to, from, and/or within a portion of the growth environment 720, for example, through one or more interface 718. In some embodiments, the processor 717 can record the intensity of each signal 770 as data, enabling real-time analysis of growth environment 720 performance. In some further embodiments, a signal 770 can be relayed to the processor 717 through a wire, Wi-Fi, BLUETOOTH®, or by any other means. In some embodiments, in response to the signal 770, the processor 717 can be configured to control oxygen (O₂) content, carbon dioxide content (CO₂), temperature, humidity, and/or other environmental conditions, to, from, and/or within the growth environment 720. In some further embodiments, multiple sensors 750 can be placed throughout the growth chamber 720 and be in simultaneous communication with each other and one or more processors 717. A signal 770 can be relayed from one sensor 750 to another sensor 750 and/or the processor 717 through a wire, Wi-Fi, BLUETOOTH®, or by any other means.

Various quantities of sensors can be implemented. In some embodiments, only a single sensor suitable to electronically detect a mist concentration value may be implemented within the growth environment. In some embodiments, two or more sensors can be implemented. In some embodiments, the one or more sensor(s) 750 can be configured to monitor growth chamber conditions in real-time and to adjust growth environment conditions without interrupting mycelium growth. In some embodiments, two or more sensors (e.g., multiple sensors) can be arranged throughout the growth environment to create a network of sensors. In some embodiments, the multiple sensors 750 can be placed adjacently (e.g., horizontally and/or vertically adjacent) with respect to each other. The distance between each sensor 750 can vary depending on the desired degree of data resolution and control within the growth environment. In some embodiments, the greater the number of sensors 750 in a growth environment 720, the finer the spatial resolution of airborne mist levels in the growth environment 720 per time point. In some embodiments, the number of sensors 750 in a portion of the growth environment 720 can range from 1 to 1,000, or any integral range therebetween, or more than 1,000. Some or all of the multiple sensors 750 can be evenly spaced with respect to one another. For example, each sensor 750 can be spaced at a regular distance from another adjacent sensor 750 (e.g., horizontal and/or vertically adjacent) to create a lattice-like network of sensors. Some or all of the multiple sensors 750 can be irregularly spaced throughout a growth environment 720. In some embodiments, a quantity of sensors can be implemented per unit area or volume to form a “sensor density.” For example, the sensor density can be with respect to the quantity of sensors per unit surface area or volume of a portion of the growth environment 720, per unit surface area or volume of mycelial growth, per unit surface area or volume of a shelving unit, per unit surface area or volume of a rack, per unit surface are or volume of a sensor 750, or per unit surface area or volume of any other physical space or object within or comprising a portion of the growth environment 720. For example, a greater density of sensors can be placed in a first desired portion of the growth environment 720 relative to a decreased density of sensors in another portion of the growth environment 720. This configuration can provide finer data resolution in said first portion of the growth environment 720. In some further embodiments, airborne mist level measurements can be recorded by multiple sensors 750 simultaneously. asynchronously, and/or combinations thereof at different times in a process.

The processor 717 can be configured to control (e.g., increase, decrease, or maintain without change) an environmental condition within a portion of the growth environment 720 to a desired environmental condition value. For example, the processor 717 can be configured to increase an environmental condition, such as the amount of airborne mist, within a portion of the growth environment 720, when the airborne mist concentration value is below a first desired value. The processor 717 can be configured to decrease an environmental condition, such as the amount of airborne mist, within a portion of the growth environment 720, when the airborne mist concentration is above a second desired value. For example, the amount of airborne mist may be decreased within a portion of the growth environment if there is too much airborne mist in a portion of the growth environment for a particular stage in a growth cycle, for example, if a stage of the growth cycle has been reached where such mist offers no or little advantage. In one embodiment, the amount of airborne mist may be decreased within a portion of the growth environment by pausing and/or stopping mist input into the growth environment and allowing mist droplets to settle from the air due to gravity. In one embodiment, the mist may be decreased when various non-desirable physical features are observed on the aerial mycelium and the level of mist in accordance with the sensor is perceived to be at an excessive level.

The first environmental condition value and the second desired environmental condition value can be the same or different value relative to each other. In some embodiments, processor 717 can be configured to control by taking no action. A growth environment condition can be maintained by preventing any substantial change in an environmental condition value or window of values between two or more time points. In this way, an environmental condition can be maintained when it is approximately equal to or at a desired environmental condition value, or at or between a first desired value (e.g., lower threshold) and a second desired value (e.g., upper threshold).

In order to provide such control, the processor 717 can generate a processor output 780 to control another component of system 710, such as an interface 718. The growth environment 720 can comprise one or more interfaces 718. An interface 718 can be configured to increase, decrease, or maintain without change an environmental condition within a portion of the growth environment 720 in response to the output 780 received from processor 717. For example, the processor 717 can be configured to control (e.g., change, or maintain constant) an airborne mist concentration level or other environmental conditions within a desired region of the growth environment 720, in response to signal 770. The interface 718 can include one or more inputs and/or outputs to adjust one or more environmental conditions. For example, one or more interfaces 718 can increase the amount of airborne mist within a portion growth environment, for example, by introducing aqueous mist into the growth environment through one or more growth environment inlet(s). It will be understood that the inlet(s) can be positioned anywhere within, or associated with, the growth environment suitable for the desired misting results, such as proximate to an outer portion (e.g., sidewall) of the growth environment as shown, e.g., within an air handling unit associated with the growth environment, or closer to the growth matrix (e.g., closer to or mounted on the growing shelves or racks). One or more interfaces 718 can decrease the amount of airborne mist within a portion or the entirety of the growth environment 710, for example, by removing aqueous mist from a desired portion of the growth environment through a growth environment outlet. Alternatively, the amount of airborne mist within a portion of the growth environment can be decreased, for example, by pausing and/or stopping mist entry into the growth chamber through one or more interfaces 718 and allowing airborne mist droplets to settle from the air column due to gravity. The amount of airborne mist within a portion the growth environment can also be adjusted, for example, by managing the water carrying capacity of the air (e.g., warmer air at the inlet), introducing temperature gradients (e.g., warm air/cooler air and surfaces), using air pressure gradients to localize temperature air temperature change (e.g., expanding air cools, air velocity creates pressure gradients), and/or other ways.

The inlets and outlets can comprise the same component, or can comprise different, separate components. In some embodiments, a plurality of sensors 750 is located in the growth environment, and it will be understood by one skilled in the art that the sensors 750 can be placed anywhere within the growth environment 720 suitable to provide the functionality described herein. In some embodiments, the sensors 750 are placed at the extremes and center of the growth environment 720, as shown in FIG. 7. In some embodiments, the sensors 750 are placed in the center of the growth environment 720 to allow broad measures of the growth environment 720 as a whole. In some further embodiments, the sensors 750 can be placed in non-central locations in the growth chamber to determine growth environment 720 performance in specific geographic areas. In some further embodiments, the sensors 750 can be placed outside of the growth environment 720. For example, the sensor can be configured to detect a mist concentration value through an exterior wall or window of the growth environment 720. In some further embodiments, the sensors 750 can be placed at different heights with respect to the growth environment 720 floor. In some further embodiments, one or more sensors 750 can be located along (e.g., on) one or more shelf 730 or shelving units or along (e.g., on) one or more racks 740. For example, a plurality of sensors 750 can be spaced (e.g., equally spaced), at two or more different longitudinal locations along a single shelf 730, as shown. A plurality of sensors can be spaced collinearly along a shelf e.g. (horizontally), and/or between shelves (e.g., vertically). A plurality of sensors 750 can be positioned at a common longitudinal location, but spaced (e.g., equally spaced) transversely across to two or more shelves 730. As shown, a plurality of three sensors 750 can be positioned such that a first sensor is positioned at an outer portion (e.g., edge on the left hand side as shown) of a first shelf 730, a second sensor is positioned between two shelves 730, and a third sensor is positioned at an opposed outer portion (e.g., edge on the right hand side as shown) of a second shelf 730. In this way, a plurality of sensors 730 can be positioned and spaced longitudinally and transversely across a plurality of shelves to form a two-dimensional sensor matrix. A two-dimensional sensor matrix can extend between two shelves on the same rack, or between two shelves, with each shelf on a different corresponding rack. A two-dimensional matrix can be extended along a third dimension (e.g., applied to multiple rows of shelves in multiple racks at different vertical heights), to form a three-dimensional sensor matrix. In this way, the system 710 can include a plurality of sensors that form a multi-dimensional matrix within the growth environment.

### Scope of Disclosure

It will be understood that although the present disclosure is discussed within the context of food and textiles, the embodiments described herein can be implemented in food, non-food, or other non-textile applications. Additionally, the following non-exhaustive list provides additional fungal genera and species which may be implemented, if not otherwise inconsistent with the present disclosure:

### Implementations

In some aspects, the present disclosure provides for an aerial mycelium, and for methods of making an aerial mycelium, wherein the aerial mycelium is a growth product of a fungus. In some embodiments, the fungus is a species of the genus Agrocybe, Albatrellus, Armillaria, Agaricus, Bondarzewia, Cantharellus, Cerioporus, Climacodon, Cordyceps, Fistulina, Flammulina, Fomes, Fomitopsis, Fusarium, Grifola, Hericium, Hydnum, Hypomyces, Hypsizygus, Ischnoderma, Laetiporus, Laricifomes, Lentinula, Lentinus, Lepista, Meripilus, Morchella, Ophiocordyceps, Panellus, Piptoporus, Pleurotus, Polyporus, Pycnoporellus, Rhizopus, Schizophyllum, Stropharia, Tuber, Tyromyces, Wolfiporia, Ceriporiopsis, Chlorociboria, Daedalea, Daedaleopsis, Daldinia, Ganoderma, Hypoxylon, Inonotus, Lenzites, Omphalotus, Oxyporus, Phanerochaete, Phellinus, Polyporellus, Porodaedalea, Pycnoporus, Scytalidium, Stereum, Trametes or Xylaria.

In some further embodiments, the fungus is a species of the genus Bondarzewia, Ceriporiopsis, Daedalea, Daedaleopsis, Fomitopsis, Ganoderma, Inonotus, Lenzites, Omphalotus, Oxyporus, Phellinus, Polyporellus, Polyporus, Porodaedalea, Pycnoporus, Stereum, Trametes or Xylaria. In some more particular embodiments, the fungus is selected from the group consisting of Bondarzewia berkleyii, Daedalea quercina, Daedaleopsis spp., Daedaleopsis confragosa, Daedaleopsis septentrionalis, Fomitopsis spp., Fomitopsis cajanderi, Fomitopsis pinicola, Ganoderma spp., Ganoderma amboinense, Ganoderma applanatum, Ganoderma atrum, Ganoderma ibbose, Ganoderma ibbose, Ganoderma capense, Ganoderma carnosum, Ganoderma cochlear, Ganoderma colossus, Ganoderma curtisii, Ganoderma donkii, Ganoderma formosanum, Ganoderma gibbosum, Ganoderma hainanense, Ganoderma hoehnelianum Ganoderma japonicum, Ganoderma lingzhi, Ganoderma lobatum, Ganoderma lucidum, Ganoderma multipileum, Ganoderma oregonense, Ganoderma pfeifferi, Ganoderma resinaceum, Ganoderma sessile, Ganoderma sichuanense, Ganoderma sinense, Ganoderma tropicum, Ganoderma tsugae, Ganoderma tuberculosum, Ganoderma weberianum, Inonotus spp., Inonotus obliqus, Inonotus hispidus, Inonotus dryadeus, Inonotus tomentosus, Lenzites betulina, Phellinus spp., Phellinus ignarius, Phellinus gilvus, Polyporus spp., Polyporus squamosus, Polyporus badius, Polyporus umbellatus, Polyporus squamosus, Polyporus tuberaster, Polyporus arcularius, Polyporus albeolaris, Polyporus radicatus, Porodaedalea pini, Pycnoporus spp., Pycnoporus spp., Pycnoporus sanguineus, Pycnoporus cinnabarinus, Stereum spp., Stereum ostea, Stereum hirsutum, Trametes spp., Trametes versicolor, Trametes elegans, Trametes suaveolens, Trametes hirsute, Trametes ibbose, Trametes ochraceae, Trametes villosa, Trametes cubensis and Trametes pubescens.

In some other embodiments, the fungus is a pigment-producing fungus of a genus selected from the group consisting of Chlorociboria, Daldinia, Hypoxylon, Phanerochaete and Scytalidium.

In yet some other embodiments, the fungus is a species of the genus Ganoderma. In some further embodiments, the fungus is Ganoderma spp., Ganoderma amboinense, Ganoderma applanatum, Ganoderma atrum, Ganoderma australe, Ganoderma brownii, Ganoderma capense, Ganoderma carnosum, Ganoderma cochlear, Ganoderma colossus, Ganoderma curtisii, Ganoderma donkii, Ganoderma formosanum, Ganoderma gibbosum, Ganoderma hainanense, Ganoderma hoehnelianum Ganoderma japonicum, Ganoderma lingzhi, Ganoderma lobatum, Ganoderma lucidum, Ganoderma multipileum, Ganoderma oregonense, Ganoderma pfeifferi, Ganoderma resinaceum, Ganoderma sessile, Ganoderma sichuanense, Ganoderma sinense, Ganoderma tropicum, Ganoderma tsugae, Ganoderma tuberculosum or Ganoderma weberianum

### Food Implementation

In yet some other embodiments, the fungus is a species of the genus Agrocybe, Albatrellus, Armillaria, Agaricus, Bondarzewia, Cantharellus, Cerioporus, Climacodon, Cordyceps, Fistulina, Flammulina, Fomes, Fomitopsis, Fusarium, Grifola, Hericium, Hydnum, Hypomyces, Hypsizygus, Ischnoderma, Laetiporus, Laricifomes, Lentinula, Lentinus, Lepista, Meripilus, Morchella, Ophiocordyceps, Panellus, Piptoporus, Pleurotus, Polyporus, Pycnoporellus, Rhizopus, Schizophyllum, Stropharia, Tuber, Tyromyces or Wolfiporia.

In some further embodiments, the fungus is a species of the genus Pleurotus. In some more particular embodiments, the fungus is Pleurotus albidus, Pleurotus citrinopilleatus, Pleurotus columbinus, Pleurotus cornucopiae, Pleurotus dryinus, Pleurotus djamor, Pleurotus eryngii, Pleurotus floridanus, Pleurotus nebrodensis, Pleurotus ostreatus, Pleurotus populinus, Pleurotus pulmonarius, Pleurotus sajor-caju, Pleurotus salmoneo-stramineus, Pleurotus salmonicolor or Pleurotus tuber-regium.

The various illustrative logics, logical blocks, modules, circuits and algorithm steps described in connection with the implementations disclosed herein may be implemented as electronic hardware, computer software, or combinations of both. The interchangeability of hardware and software has been described generally, in terms of functionality, and illustrated in the various illustrative components, blocks, modules, circuits and steps described above. Whether such functionality is implemented in hardware or software depends upon the particular application and design constraints imposed on the overall system.

The hardware and data processing apparatus used to implement the various illustrative logics, logical blocks, modules and circuits described in connection with the aspects disclosed herein may be implemented or performed with a general-purpose single- or multi-chip processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general-purpose processor may be a microprocessor, or, any conventional processor, controller, microcontroller, or state machine. A processor also may be implemented as a combination of computing devices, such as a combination of a DSP and a microprocessor, a plurality of microprocessors, one or more microprocessors in conjunction with a DSP core, or any other such configuration. In some implementations, particular steps and methods may be performed by circuitry that is specific to a given function.

In one or more aspects, the functions described may be implemented in hardware, digital electronic circuitry, computer software, firmware, including the structures disclosed in this specification and their structural equivalents thereof, or in any combination thereof. Implementations of the subject matter described in this specification also can be implemented as one or more computer programs, i.e., one or more modules of computer program instructions, encoded on a computer storage media for execution by, or to control the operation of, data processing apparatus.

If implemented in software, the functions may be stored on or transmitted over as one or more instructions or code on a computer-readable medium. The steps of a method or algorithm disclosed herein may be implemented in a processor-executable software module which may reside on a tangible, non-transitory computer-readable medium. Computer-readable media includes both computer storage media and communication media including any medium that can be enabled to transfer a computer program from one place to another. A storage media may be any available media that may be accessed by a computer.

A software module may reside in random access memory (RAM), flash memory, read only memory (ROM), electrically programmable ROM (EPROM), electrically erasable programmable ROM (EEPROM), registers, hard disk, a removable disk, a CD ROM, or any other form of storage medium known in the art. A storage medium is coupled to the processor such that the processor can read information from, and write information to, the storage medium. In the alternative, the storage medium may be integral to the processor. Disk and disc, as used herein, includes compact disc (CD), laser disc, optical disc, digital versatile disc (DVD), floppy disk and blue ray disc where disks usually reproduce data magnetically, while discs reproduce data optically with lasers. Combinations of the above should also be included within the scope of computer readable media. The processor and the storage medium may reside in an ASIC. The ASIC may reside in a user terminal. In the alternative, the processor and the storage medium may reside as discrete components in a user terminal.

Additionally, the operations of a method or algorithm may reside as one or any combination or set of codes and instructions on a machine readable medium and computer-readable medium, which may be incorporated into a computer program product.

Various modifications to the implementations described in this disclosure may be readily apparent to those skilled in the art, and the generic principles defined herein may be applied to other implementations without departing from the spirit or scope of this disclosure. Thus, the claims are not intended to be limited to the implementations shown herein, but are to be accorded the widest scope consistent with this disclosure, the principles and the novel features disclosed herein. Additionally, a person having ordinary skill in the art will readily appreciate, the terms “upper” and “lower” are sometimes used for ease of describing the figures, and indicate relative positions corresponding to the orientation of the figure on a properly oriented page, and may not reflect the proper orientation of a feature as implemented.

While certain embodiments of the inventions have been described, these embodiments have been presented by way of example only and are not intended to limit the scope of the disclosure. Indeed, the novel methods and systems described herein may be embodied in a variety of other forms. Furthermore, various omissions, substitutions and changes in the systems and methods described herein may be made without departing from the spirit of the disclosure. The accompanying claims and their equivalents are intended to cover such forms or modifications as would fall within the scope and spirit of the disclosure. Accordingly, the scope of the present inventions is defined only by reference to the appended claims.

Features, materials, characteristics, or groups described in conjunction with a particular aspect, embodiment, or example are to be understood to be applicable to any other aspect, embodiment or example described in this section or elsewhere in this specification unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The protection is not restricted to the details of any foregoing embodiments. The protection extends to any novel one, or any novel combination, of the features disclosed in this specification (including any accompanying claims, abstract and drawings), or to any novel one, or any novel combination, of the steps of any method or process so disclosed.

The word “exemplary” is used herein to mean “serving as an example, instance, or illustration.” Any aspect or embodiment described herein as “exemplary” is not necessarily to be construed as preferred or advantageous over other aspects or embodiments. Various aspects of the novel systems, apparatuses, and methods are described more fully hereinafter with reference to the accompanying drawings. This disclosure may, however, be embodied in many different forms and should not be construed as limited to any specific structure or function presented throughout this disclosure. Rather, these aspects are provided so that this disclosure will be thorough and complete, and will fully convey the scope of the disclosure to those skilled in the art. Based on the teachings herein one skilled in the art should appreciate that the scope of the disclosure is intended to cover any aspect of the novel systems, apparatuses, and methods disclosed herein, whether implemented independently of, or combined with, any other aspect described. For example, an apparatus may be implemented or a method may be practiced using any number of the aspects set forth herein. In addition, the scope of the disclosure is intended to cover such an apparatus or method which is practiced using other structure, functionality, or structure and functionality in addition to or other than the various aspects of the disclosures set forth herein. It should be understood that any aspect disclosed herein may be embodied by one or more elements of a claim.

Furthermore, certain features that are described in this disclosure in the context of separate implementations can also be implemented in combination in a single implementation. Conversely, various features that are described in the context of a single implementation can also be implemented in multiple implementations separately or in any suitable subcombination. Although features may be described above as acting in certain combinations, one or more features from a claimed combination can, in some cases, be excised from the combination, and the combination may be claimed as a subcombination or variation of a subcombination.

The features and attributes of the specific embodiments disclosed above may be combined in different ways to form additional embodiments, all of which fall within the scope of the present disclosure. Also, the separation of various system components in the implementations described above should not be understood as requiring such separation in all implementations, and it should be understood that the described components and systems can generally be integrated together in a single product or packaged into multiple products.

Moreover, while operations may be depicted in the drawings or described in the specification in a particular order, such operations need not be performed in the particular order shown or in sequential order, or that all operations be performed, to achieve desirable results. Other operations that are not depicted or described can be incorporated in the example methods and processes. For example, one or more additional operations can be performed before, after, simultaneously, or between any of the described operations. Further, the operations may be rearranged or reordered in other implementations. Those skilled in the art will appreciate that in some embodiments, the actual steps taken in the processes illustrated and/or disclosed may differ from those shown in the figures. Depending on the embodiment, certain of the steps described above may be removed, others may be added. Furthermore, the features and attributes of the specific embodiments disclosed above may be combined in different ways to form additional embodiments, all of which fall within the scope of the present disclosure.

For purposes of this disclosure, certain aspects, advantages, and novel features are described herein. Not necessarily all such advantages may be achieved in accordance with any particular embodiment. Thus, for example, those skilled in the art will recognize that the disclosure may be embodied or carried out in a manner that achieves one advantage or a group of advantages as taught herein without necessarily achieving other advantages as may be taught or suggested herein.

Conditional language, such as “can,” “could,” “might,” or “may,” unless specifically stated otherwise, or otherwise understood within the context as used, is generally intended to convey that certain embodiments include, while other embodiments do not include, certain features, elements, and/or steps. Thus, such conditional language is not generally intended to imply that features, elements, and/or steps are in any way required for one or more embodiments or that one or more embodiments necessarily include logic for deciding, with or without user input or prompting, whether these features, elements, and/or steps are included or are to be performed in any particular embodiment.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z. Thus, as used herein, a phrase referring to “at least one of X, Y, and Z” is intended to cover: X, Y, Z, X and Y, X and Z, Y and Z, and X, Y and Z.

The headings provided herein, if any, are for convenience only and do not necessarily affect the scope or meaning of the devices and methods disclosed herein.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount.

The scope of the present disclosure is not intended to be limited by the specific disclosures of embodiments in this section or elsewhere in this specification and may be defined by claims as presented in this section or elsewhere in this specification or as presented in the future. The language of the claims is to be interpreted broadly based on the language employed in the claims and not limited to the examples described in the present specification or during the prosecution of the application, which examples are to be construed as non-exclusive.

1. A method for growing mycelium, comprising the steps of:
providing a growth matrix within a growth environment, the growth matrix comprising a growth medium and a fungus; and
electronically detecting an airborne mist concentration value within a portion of the growth environment.

2. The method of claim 1, wherein the portion of the growth environment comprises between 0.01% to 100% of the growth environment total volume.

3. The method of claim 1, further comprising controlling an airborne mist concentration level within the portion of the growth environment in response to the airborne mist concentration value.

4. The method of claim 3, wherein controlling comprises at least one of:
increasing an amount of airborne mist within the portion of the growth environment when the airborne mist concentration value is below a first desired value; and
decreasing an amount of airborne mist within the portion of the growth environment when the airborne mist concentration value is above a second desired value.

5. The method of claim 4, wherein increasing the amount of airborne mist within the portion of the growth environment comprises introducing mist into the portion of the growth environment through a growth environment inlet, and decreasing the amount of airborne mist within the portion of the growth environment comprises at least one of:
(1) stopping introduction of airborne mist into the portion of the growth environment during the introducing step;
(2) reducing a rate of mist being introduced into the portion of the growth environment during the introducing step;
(3) pausing the introduction of airborne mist into the portion of the growth environment during the introducing step; and
(4) removing mist from the portion of the growth environment through a growth environment outlet.

6. The method of claim 4, wherein the first desired value and the second desired value are different values relative to each other.

7. The method of claim 6, wherein controlling comprises maintaining an amount of airborne mist within the portion of the growth environment when the airborne mist concentration value is at or between the first desired value and the second desired value.

8. The method of claim 1, wherein electronically detecting comprises detecting the airborne mist concentration value with at least one sensor located within the portion of the growth environment.

9. The method of claim 8, wherein electronically detecting comprises detecting the airborne mist concentration value from a plurality of sensors located in one or more portions of the growth environment.

10. The method of claim 8, wherein the at least one sensor comprises a fog sensor.

11. The method of claim 9, wherein the fog sensor is configured to detect electromagnetic radiation comprising a wavelength greater than or equal to 380 nm and less than or equal to 2,800 nm.

12. An apparatus for growing mycelium, comprising:
a growth environment, wherein the growth environment comprises a total volume;
a portion of the growth environment configured to grow mycelium from a growth matrix, the growth matrix comprising a growth medium and a fungus, wherein the portion of the growth environment can comprise from 0.01% to 100% of the total volume;
at least one sensor configured to generate a first signal indicative of an airborne mist concentration value within the portion of the growth environment, and electronically communicate the first signal to a processor.

13. The apparatus of claim 12, comprising the processor, wherein the processor is configured to receive the first signal and control an airborne mist concentration level within the portion of the growth environment in response to the first signal.

14. The apparatus of claim 13, wherein the processor is further configured to at least one of:
increase an amount of airborne mist within the portion of the growth environment when the airborne mist concentration value is below a first desired value; and
decrease an amount of airborne mist within the portion of the growth environment when the airborne mist concentration value is above a second desired value.

15. The apparatus of claim 12, further comprising: at least one inlet configured to introduce aqueous mist into the portion of the growth environment.

16. The apparatus of claim 15, wherein the at least one inlet comprises a mister.

17. The apparatus of claim 12, wherein the at least one sensor comprises one of a near-infrared fog sensor, an infrared fog sensor, or a visible light fog sensor.

18. The apparatus of claim 12, wherein the at least one sensor comprises a source of electromagnetic radiation and a phototransistor that is tuned to respond to a desired electromagnetic wavelength.

19. The apparatus of claim 12, wherein the at least one sensor comprises a plurality of sensors.

20. The apparatus of claim 19, wherein the plurality of sensors form a multi-dimensional matrix of sensors.
